(19) United States
(12) Plant Patent Application Publication
(10) Pub. No.: US 2015/0313049 P1
(43) Pub. Date: Oct. 29, 2015

(54) Rose plant named 'RUIPE0306C'

(50) ;

(71) Applicant: De Ruiter Intellectual Property B.V., Amstelveen (NL)

(72) Inventor: Hendrikus C.A. De Groot, Amstelveen (NL)

(73) Assignee: De Ruiter Intellectual Property B.V., Amstelveen (NL)

(21) Appl. No.: 14/120,945

(22) Filed: Jul. 14, 2014

(30) Foreign Application Priority Data
Apr. 28, 2014 (QZ) 2014/1150

(51) Int. Cl. A01H 5/00

(52) U.S. Cl. PLT/136

(57) ABSTRACT

A new Rose plant particularly distinguished by having orange flowers and a mostly upright form, is disclosed.

### GENUS AND SPECIES

Rosa L.

### VARIETY DENOMINATION

‘RUIPE0306C’

### CROSS REFERENCE TO RELATED APPLICATIONS

This application claims, under 35 U.S.C. § 119, priority to and the benefit of European Community Plant Variety Rights Application No. 2014/1150, filed Apr. 28, 2014, the entire contents of which are incorporated herein by reference for all purposes.

### BACKGROUND OF THE NEW PLANT

The present invention comprises a new and distinct variety of Rose, botanically known as Rosa L., and hereinafter referred to by the variety name ‘RUIPE0306C’. ‘RUIPE0306C’ originated from a cross conducted in May 2004 in Hazerswoude, The Netherlands between the proprietary female Rosa variety ‘P-01-0152-001’ (unpatented), and the proprietary male Rosa variety ‘Ruira013a’ (unpatented).

The seeds from the cross were sown and plants were grown for evaluation in a greenhouse without heating. An individual plant designated ‘RUIPE0306C’ was selected in January 2006. ‘RUIPE0306C’ was first vegetatively propagated in May 2006 in Hazerswoude, The Netherlands by cuttings. ‘RUIPE0306C’ was found to reproduce true to type in successive generations of asexual propagation via cuttings.

### SUMMARY

The following are the most outstanding and distinguishing characteristics of this new variety when grown under normal horticultural practices in Hazerswoude, The Netherlands.

1. Orange flowers; and

2. A mostly upright form.

### DESCRIPTION OF THE PHOTOGRAPHS

This new Rose plant is illustrated by the accompanying photographs which show the plant's overall plant habit including form, foliage, buds, and flowers. The colors shown are as true as can be reasonably obtained by conventional photographic procedures. FIG. 1 is of 24-week-old plant grown indoors in Amstelveen, The Nertherlands and taken in January 2014 and FIG. 2 is of the plants parts taken from the 24-week-old plant.

FIG. 1 shows the overall plant habit, including the blooms, buds, and foliage, in a pot.

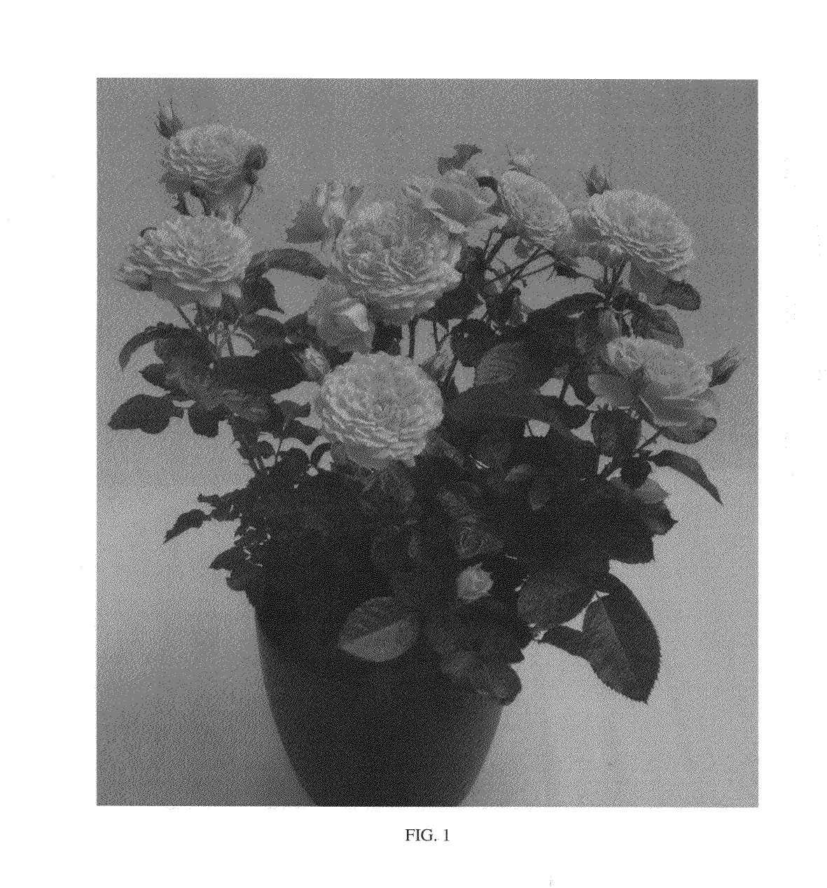

FIG.2 shows a close-up of individual flower parts of the plant, where:

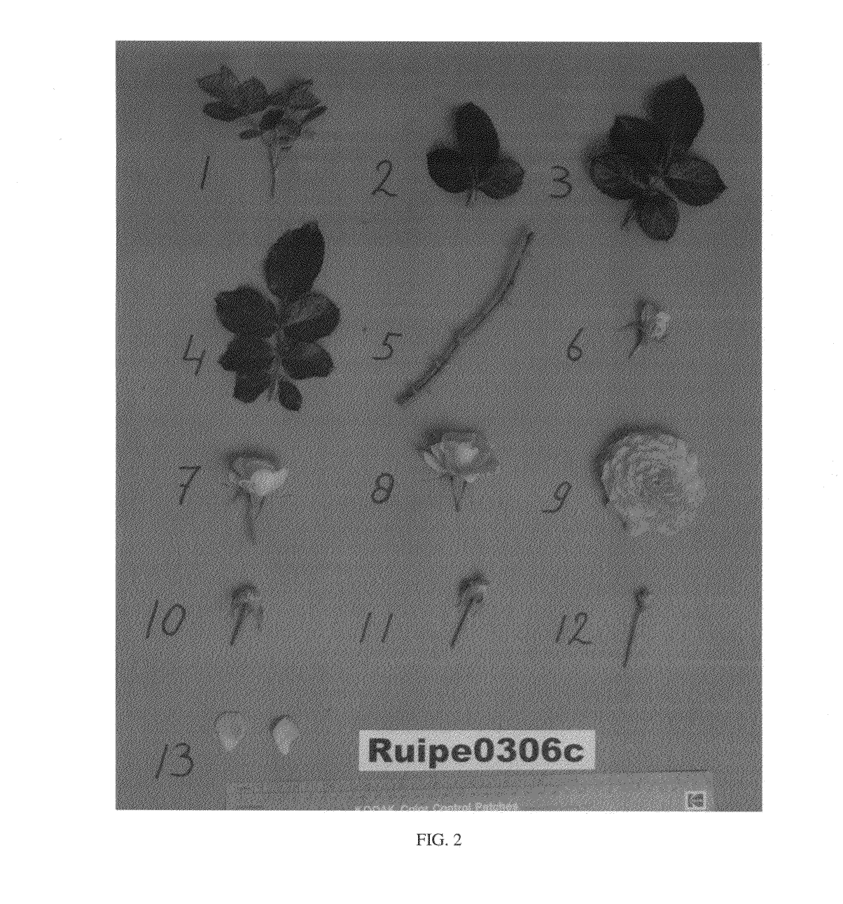

1 shows immature leaves and leaflets.

2 shows a leaf having 3 leaflets.

3 shows a leaf having 5 leaflets.

4 shows a leaf having 7 leaflets.

5 shows a portion of a stem with thorns

6 show a side view of a flower in immature bloom

7 shows a side view of a flower in immature bloom.

8 shows a side view of a flower in immature bloom.

9 shows a mature flower in bloom.

10 shows a side view of the sepals and reproductive organs after bloom.

11 shows a side view of the sepals and reproductive organs after bloom.

12 shows a side view of the reproductive organs without the sepals.

13 shows the upper and lower surfaces of individual petals.

### DESCRIPTION OF THE NEW VARIETY

The following detailed descriptions set forth the distinctive characteristics of ‘RUIPE0306C’. The data which define these characteristics were collected from 24-week-old plants grown in a glasshouse in Amstelveen, The Netherlands in January 2014. Color references are to The R.H.S. Colour Chart of The Royal Horticultural Society of London (R.H.S.), 1995.
Classification:
   Family.—Rosaceae.
   Botanical.—Rosa L.
   Designation.—‘RUIPE0306C’.
   Parentage.—Female parent: The proprietary female rose variety ‘P-01-0152-001’ (unpatented) Male parent: The proprietary male rose variety ‘Ruira013a’ (unpatented).
Plant:
   Height.—30.0 cm to 40.0 cm.
   Spread.—30.0 cm.
   Form.—Mostly upright.
   Time to produce a rooted cutting.—Average is 4 weeks.
   Time to bloom from propagation.—Average is 10 weeks.
   Lastingness of inflorescence on the plant.—Average is 3 weeks.
Lateral branches/peduncles:
   Color.—RHS 138A.
   Pubescence.—Absent.
   Stem shape and appearance.—Rounded and glossy.
   Diameter.—Average is 0.4 cm.
   Length.—Average is 20.0 cm.
   Internode length.—Average is 3.0 cm.
   Anthocyanin.—Absent.
   Thorns.—Density (quantity per unit area): Average is 13 to 15 thorns per stem Shape: Concave Color: RHS 163C Length: 1.2 cm Width (at the base): Average is 0.7 cm.
Foliage:
   Arrangement and type.—Compound, imparipinnate.
   Quantity of leaflets per leaf.—3, 5, or 7 leaflets per leaf.
   Leaf length.—Average is 12.0 cm.
   Leaf width.—Average is 11.0 cm.
   Terminal leaflet.—Length: 5.5 cm Width: 3.5 cm Shape: Ovate Apex: Acute Base: Round Margin: Serrate Color, immature leaf: Upper surface: RHS 137B Lower surface: RHS 200C Color, mature leaf: Upper surface: RHS 139A Lower surface: RHS 147B Texture: Upper surface: Glabrous, leathery Lower surface: Glabrous.
   Lateral leaflet.—Length: Average is 5.0 cm Width: Average is 3.4 cm Shape: Ovate Apex: Acute Base: Round Margin: Serrate Color, immature leaf: Upper surface: RHS 137B Lower surface: RHS 200C Color, mature leaf: Upper surface: RHS 139A Lower surface: RHS 147B Texture: Upper surface: Glabrous, leathery Lower surface: Glabrous.
   Leaflet venation pattern.—Pinnate.
   Leaflet venation color.—Upper surface: RHS 176B Lower surface: RHS 147C.
   Petioles, leaves.—Length: 1.2 cm Diameter: 0.1 cm Color: RHS 147A Texture: Pubescent Thorns: Present; deep concave in shape and 0.6 cm in length.
   Petioles, leaflets.—Length: 0.1 cm Diameter: 0.1 cm Color: RHS 138B Texture: Pubescent Thorns: Absent.
   Stipules.—Length: average: 0.8 cm Width: average: 0.4 cm Appearance: Scale-like Apex: Auricles about 0.5 cm in length and 0.1 cm in width Base: Normal Margin: Entire Color, immature leaf: Upper surface: RHS 138B Lower surface: RHS 137C Color, mature leaf: Upper surface: RHS 138B Lower surface: RHS 137C Texture (both upper and lower surfaces): Smooth.
Flowers or inflorescence:
   Appearance and arrangement.—Flowers are born single on peduncle, sometimes in a large spray, there are 3 to 4 flowers.
   Fragrance.—Very light.
   Diameter.—Average is 7.0 cm.
   Depth or height.—Average is 4.0 cm.
   Lastingness of flowers on the plant.—Average is 14 to 16 days.
   Buds.—Length: Average is 2.5 cm Width: Average is 1.5 cm Shape: Ovate Color: RHS 31B.
   Petals.—Quantity per flower: Average is 50 petals Length: Average is 2.8 cm Width: Average is 2.5 cm Shape: Shell-shaped Apex: Mucronulate Base: Cuneate Margin: Reflexing Color, when opening: Upper surface: RHS 30D Lower surface: RHS 28D Color, when fully open: Upper surface: RHS 30D Lower surface: RHS 28D Surface texture (both upper and lower surfaces): Velvety Petaloids: Absent.
   Sepals.—Quantity per flower: 5 Length: 27.0 mm Width: 9.0 mm Shape: Ensiform Apex: Pungent Base: Truncate Margin: Ciliate Color: Upper surface: Between RHS 139B and RHS 139C Lower surface: RHS 138A.
   Surface texture.—Upper surface: Pubescent Lower surface: Pubescent.
Reproductive organs:
   Stamens.—Quantity per flower: Average is 85 Anther color: RHS 22C Anther shape: Bi-lobate Filament length: 0.5 cm.
   Pistils.—Quantity per flower: Average is 13 Pistil length: Average is 0.4 cm Stigma shape: Fan-shaped Stigma color: RHS 163C Style length: Average is 0.6 cm Style color: RHS 43A Receptacle: Height: 1.3 cm Diameter: 1.5 cm Color: RHS 144B Texture: smooth.
Fruit and seed set: None observed.
Disease and pest/insect resistance: Mildrew resistant, insect resistance not observed.

### COMPARISON WITH PARENTAL LINES AND KNOWN VARIETY

‘RUIPE0306C’ is a distinct variety of Rose and is distinguished from its parents as described in Table 1.

TABLE 1
Comparison with Parental Lines

| | | Female Parent | Male Parent |
|---|---|---|---|
| Characteristic | ‘RUIPE0306C’ | ‘P-01-0152-001’ | ‘Ruira013a’ |
| Flower color | Orange | Orange-red | Orange-pink |

When ‘RUIPE0306C’ is compared to the commercial variety ‘RUIPE0306D’, the following differences are described in Table 2.

TABLE 2
Comparison with Similar Variety

| Characteristic | ‘RUIPE0306C’ | ‘RUIPE0306D’ |
|---|---|---|
| Flower color | Orange | Orange-pink |
| Average number of | 50 | 30 to 35 |
| petals per flower | | |

I claim:

1. A new and distinct variety of Rose plant designated ‘RUIPE0306C’ as shown and described herein.
